(19) United States
(12) Patent Application Publication
(10) Pub. No.: US 2018/0325862 A1
(43) Pub. Date: Nov. 15, 2018

(54) METHODS FOR TREATING ALZHEIMER?S DISEASE AND RELATED DISORDERS

(71) Applicant: AZTherapies, Inc., Boston, MA (US)

(72) Inventor: David R. ELMALEH, Newton, MA (US)

(73) Assignee: AZTherapies, Inc., Boston, MA (US)

(21) Appl. No.: 15/777,069

(22) PCT Filed: Nov. 21, 2016
(86) PCT No.: PCT/US16/63143

§ 371 Date: May 17, 2018

Related U.S. Application Data
(60) Provisional application No. 62/257,616, filed on Nov. 19, 2015.

(51) Int. Cl. A61K 31/352; A61K 31/192; A61P 25/28

(52) U.S. Cl. CPC A61K 31/352; A61P 25/28; A61K 31/192

(57) ABSTRACT

The invention is directed to a method of treating Alzheimer's Disease by administering to a subject in need thereof a therapeutically effective amount of cromolyn and optionally ibuprofen. The cromolyn may be in the form of cromolyn sodium and administered by inhalation.

### CROSS REFERENCE TO RELATED APPLICATIONS

This application claims the benefit of U.S. Ser. No. 62/257,616, filed on Nov. 19, 2015, which is incorporated herein by reference.

### BACKGROUND OF THE INVENTION

Alzheimer's disease (AD) is an irreversible, progressive brain disease with an average course of eight to twenty years. The disease results in cognitive and functional impairment, which may affect memory, thinking skills, orientation, personality, and in its most severe form, the ability to carry on the most basic tasks of daily life. AD is the sixth leading cause of death in the United States Alzheimer's and dementia are part of diseases resulting from a complex neurodegenerative mechanism associated with the process of aging genetic mutation or brain injury.

An estimated 5.4 million Americans have AD. It is estimated that one in eight people over 65 years and almost half of persons 85 years and older have AD. However, because AD is under-diagnosed, more than half of afflicted persons are not identified as Alzheimer's patients and are not being treated for the disease.

By 2030, the segment of the U.S. population aged 65 and older is expected to double as a result of the aging of the “baby-boomer” generation and result in a doubling of the number of Alzheimer's disease sufferers.

According to Alzheimer's Disease International's 2015 World Alzheimer's Report, an estimated 36 million worldwide exhibit dementia. This number is expected to double every 20 years, to 66 million by 2030 and 115 million by 2050. Alzheimer's dementia accounts for the majority of dementia and is estimated to be 50% to 75% of all dementias.

Worldwide dementia is severely underdiagnosed. Research shows that in high-income countries, only 20% to 50% of dementia cases are correctly identified and documented by primary physicians. In low to middle-income countries, this figure is much lower. One study in India suggested 90% of subjects with dementia remain unidentified. As the world's population grows older, early diagnosis and treatment will be of critical concern for improving the lives of those living with AD.

Parkinson's disease (PD, also known as idiopathic or primary Parkinsonism, hypokinetic rigid syndrome (HRS), or paralysis agitans) is a degenerative disorder of the central nervous system mainly affecting the motor system. The motor symptoms of Parkinson's disease result from the death of dopamine-generating cells in the substantia nigra, a region of the midbrain.

Amyotrophic lateral sclerosis (ALS), also known as Lou Gehrig's disease and Charcot disease, is a specific disorder that involves the death of neurons. ALS is characterized by stiff muscles, muscle twitching, and gradually worsening weakness due to muscle wasting. This results in difficulty speaking, swallowing, and eventually breathing.

Dementia with Lewy bodies (DLB), also known under a variety of other names including Lewy body dementia (LBD), diffuse Lewy body disease, cortical Lewy body disease, and senile dementia of Lewy type, is a type of dementia closely associated with Parkinson's disease. It is characterized anatomically by the presence of Lewy bodies, clumps of alpha-synuclein and ubiquitin protein in neurons, detectable in post mortem brain histology.

Vascular dementia, also known as multi-infarct dementia (MID) and vascular cognitive impairment (VCI), is dementia caused by problems in the supply of blood to the brain, typically a series of minor strokes, leading to stepwise cognitive decline. Vascular dementia is the second-most-common form of dementia after Alzheimer's disease (AD) in older adults. The term refers to a syndrome consisting of a complex interaction of cerebrovascular disease and risk factors leading to changes in the brain structures (strokes, lesions), and resulting changes in cognition.

The preclinical stage of Alzheimer's disease has frequently been termed mild cognitive impairment (MCI), but whether this term corresponds to a different diagnostic stage or identifies the first step of AD is a matter of dispute. See, Petersen R. C., “The Current Status of Mild Cognitive Impairment—What Do We Tell Our Patients?” Nat. Clin. Pract. Neurol., (2007) 3(2):60-1.

Mild cognitive impairment is a brain function syndrome involving the onset and evolution of cognitive impairments beyond those expected based on the age and education of the individual but which are not significant enough to interfere with individuals' daily activities. See, Petersen, et al., “Mild cognitive impairment: clinical characterization and outcome,” Arch. Neurol., (1999) 56 (3): 303-8. MCI is often found to be a transitional stage between normal aging and dementia. Although MCI can present with a variety of symptoms, when memory loss is the predominant symptom it is termed “amnestic MCI” (aMCI) and is frequently seen as a prodromal stage of AD. Grundman et al., “Mild cognitive impairment can be distinguished from Alzheimer disease and normal aging for clinical trials,” Arch. Neurol. (2004) 61(1): 59-66. Studies suggest that these individuals tend to progress to probable Alzheimer's disease at a rate of approximately 10% to 15% per year. (Id.)

There is evidence suggesting that although aMCI patients may not meet neuropathologic criteria for AD, patients may be in a transitional stage of evolving Alzheimer's disease; patients in this hypothesized transitional stage demonstrated diffuse amyloid in the neocortex and frequent neurofibrillary tangles in the medial temporal lobe. See, Petersen et al., “Neuropathologic features of amnestic mild cognitive impairment,” Arch. Neurol. (2006) 63 (5): 665-72.

Additionally, when individuals have impairments in domains other than memory, the condition is classified as nonamnestic single- or multiple-domain MCI and these individuals are believed to be more likely to convert to other dementias (e.g., dementia with Lewy bodies). Tabert, et al., “Neuropsychological prediction of conversion to Alzheimer disease in patients with mild cognitive impairment,” Arch Gen Psychiatry. (2006) 63(8):916-24. However, some instances of MCI may simply remain stable over time or even remit. Causation of the syndrome in and of itself remains unknown, as therefore do prevention and treatment.

The first symptoms of AD are often mistakenly attributed to aging or stress. Waldemar G., “Recommendations for the Diagnosis and Management of Alzheimer's Disease and Other Disorders Associated with Dementia: EFNS Guideline,” Eur J Neurol. (2007) 14(1):e1-26. Many subjects with genetic pre-disposition to AD risk, with no obvious symptoms, may also be identified early in the disease process. In some cases, detailed neuropsychological testing can reveal mild cognitive difficulties up to eight years before a person fulfills the clinical criteria for diagnosis of AD. Bäckman, et al., “Multiple Cognitive Deficits During the Transition to Alzheimer's Disease,” J. of Internal Medicine, (2004) 256(3):195-204. These early symptoms can affect the most complex daily living activities. Nygård L., “Instrumental Activities of Daily Living: A Stepping-stone Towards Alzheimer's Disease Diagnosis in Subjects with Mild Cognitive Impairment?” Acta Neurol Scand. (2003) Suppl(179):42-6. The most noticeable deficit is memory loss, which shows up as difficulty in remembering recently learned facts and inability to acquire new information (Backman, 2004; Arnáiz, et al., “Neuropsychological features of mild cognitive impairment and preclinical Alzheimer's disease,” Acta Neurol Scand Suppl. (2003) 179:34-41).

Subtle problems with the executive functions of attentiveness, planning, flexibility, and abstract thinking, or impairments in semantic memory (memory of meanings and concept relationships) can also be symptomatic of the early stages of AD (Backman, 2004). Apathy can be observed at this stage and remains the most persistent neuropsychiatric symptom throughout the course of the disease. Landes, et al., “Apathy in Alzheimer's Disease,” J Am Geriatr Soc. (2001) 49(12):1700-7. Depressive symptoms, irritability, and reduced awareness of subtle memory difficulties also occur commonly. Murray E. D. et al. (2012). Depression and Psychosis in Neurological Practice. In Bradley W. G. et al. Bradley's neurology in clinical practice. (6th ed.). Philadelphia, Pa.: Elsevier/Saunders.

In people with AD, the increasing impairment of learning and memory eventually leads to a definitive diagnosis. In a small portion of them, difficulties with language, executive functions, perception (agnosia), or execution of movements (apraxia) are more prominent than memory problems. Förstl, et al., “Clinical Features of Alzheimer's Disease,” European Archives of Psychiatry and Clinical Neuroscience. (1999) 249(6):288-290. AD does not affect all memory capacities equally. Older memories of the person's life (episodic memory), facts learned (semantic memory), and implicit memory (the memory of the body on how to do things, such as using a fork to eat) are affected to a lesser degree than new facts or memories. Carlesimo, et al., “Memory Deficits in Alzheimer's Patients: A Comprehensive Review,” Neuropsychol Rev. (1992) 3(2):119-69 and Jelicic, et al., “Implicit Memory Performance of Patients with Alzheimer's Disease: A Brief Review,” International Psychogeriatrics. (1995) 7(3):385-392.

Language problems are mainly characterized by a shrinking vocabulary and decreased word fluency, which lead to a general impoverishment of oral and written language. Förstl, 1999, and Taler, et al., “Language Performance in Alzheimer's Disease and Mild Cognitive Impairment: a comparative review,” J Clin Exp Neuropsychol. (2008) 30 (5):501-56. In this stage, the person with Alzheimer's is usually capable of communicating basic ideas adequately. Förstl, 1999; Taler, 2008; and Frank E. M., “Effect of Alzheimer's Disease on Communication Function,” J S C Med Assoc. (1994) 90(9):417-23. While performing fine motor tasks such as writing, drawing, or dressing, certain movement coordination and planning difficulties (apraxia) may be present, but they are commonly unnoticed. Förstl, 1999. As the disease progresses, people with AD can often continue to perform many tasks independently but may need assistance or supervision with the most cognitively demanding activities. Id.

Progressive deterioration eventually hinders independence, with subjects being unable to perform most common activities of daily living. Id. Speech difficulties become evident due to an inability to recall vocabulary, which leads to frequent incorrect word substitutions (paraphasias). Reading and writing skills are also progressively lost. Id., Frank, 1994. Complex motor sequences become less coordinated as time passes and as AD progresses, so the risk of falling increases. Förstl, 1999. During this phase, memory problems worsen, and the person may fail to recognize close relatives. Id. Long term memory, which was previously intact, becomes impaired. Id.

Behavioral and neuropsychiatric changes become more prevalent. Common manifestations are wandering, irritability, and labile affect, leading to crying, outbursts of unpremeditated aggression, or resistance to caregiving. Id. Sundowning can also appear. Volicer, et al., “Sundowning and Circadian Rhythms in Alzheimer's Disease,” Am J Psychiatry, 2001 [Retrieved 2008-08-27] 158(5):704-11. Approximately 30% of people with AD develop illusionary misidentifications and other delusional symptoms. Förstl, 1999. Subjects also lose insight of their disease process and limitations (anosognosia). Id. Urinary incontinence can develop. Id. These symptoms create stress for relatives and caretakers, which can be reduced by moving the person from home care to other long-term care facilities. Id.; Gold, et al., “When Home Caregiving Ends: A Longitudinal Study of Outcomes for Caregivers of Relatives with Dementia,” J Am Geriatr Soc. (1995) 43(1):10-6.

During the final stage of AD, the person is completely dependent upon caregivers. Förstl, 1999. Language is reduced to simple phrases or even single words, eventually leading to complete loss of speech. Id.; Frank, 1994. Despite the loss of verbal language abilities, people can often understand and return emotional signals. Förstl, 1999. Although aggressiveness can still be present, extreme apathy and exhaustion are much more common results. Id. People with AD will ultimately not be able to perform even the simplest tasks without assistance. Id. Muscle mass and mobility deteriorate to the point where they are bedridden, and they lose the ability to feed themselves. Id. AD is a terminal illness, with the cause of death typically being an external factor, such as infection of pressure ulcers or pneumonia, not the disease itself. Id.

The treatment of AD will require addressing the multiple triggers of pathogenesis. There are believed to be two primary neuropathologies in the brains of AD patients: a) extracellular protein plaques principally composed of amyloid-beta (Aβ) peptides, also known as amyloid plaques; and b) intracellular tangles of fibrils composed of tau protein found inside of neurons, also known as tau tangles. The advent and spread of neurotoxic oligomeric aggregates of Aβ is widely regarded as the key trigger leading to neuronal damage, which then leads to the accumulation of intracellular tau tangles, and finally to neuronal cell death in AD pathogenesis.

Aβ peptides (37 to 43 amino acids in length) are formed by sequential cleavage of the native amyloid precursor protein or APP. Karran et al., “The amyloid cascade hypothesis for Alzheimer's disease: an appraisal for the development of therapeutics,” Nature Reviews (2011) 10:698-712. Aberrant Aβ peptide isoforms that are 40 or 42 amino acids in length (Aβ-40/42) misfold into aggregates of oligomers that grow into fibrils to accumulate in the brain as amyloid plaques. More importantly for AD pathogenesis, the alternate fate of Aβ oligomers is to become trapped in neuronal synapses where they hamper synaptic transmission, which eventually results in neuronal degeneration and death. Haass, et al., “Soluble protein oligomers in neurodegeneration: lessons from the Alzheimer's amyloid β-peptide.” Nature Reviews Mol. Cell Biol. (2007) 8:101-112; Hashimoto et al., “Apolipoprotein E, especially Apolipoprotein E4, Increases the Oligomerization of amyloid beta Peptide,” J. Neurosci. (2012) 32:15181-15192.

The cascade of Aβ oligomer-mediated neuronal intoxication is exacerbated by another AD trigger, namely chronic local inflammatory responses in the brain. Krstic, et al., “Deciphering the mechanism underlying late-onset Alzheimer disease,” Nature Reviews Neurology, (2012):1-10. AD has a chronic neuro-inflammatory component that is characterized by the presence of abundant microglial cells associated with amyloid plaque. Heneka, et al., “Acute treatment with the PPARγ agonist pioglitazone and ibuprofen reduces glial inflammation and A 1-42 levels in APPV717I transgenic mice,” Brain (2005) 128:1442-1453; Imbimbo, et al., “Are NSAIDs useful to treat Alzheimer's disease or mild cognitive impairment,” Front. Aging Neurosci (2010) 2(article 19):1-14. These cycloxygenase (COX1/COX2)-expressing microglia, which phagocytose amyloid oligomers, become activated to secrete pro-inflammatory cytokines. Hoozemans, et al., “Soothing the Inflamed Brain: Effect of Non-Steroidal Anti-Inflammatory Drugs on Alzheimer's Disease Pathology,” CNS & Neurological Disorders—Drug Targets (2011) 10:57-67; Griffin T. S., “What causes Alzheimer's?” The Scientist (2011) 25:36-40; Krstic, 2012. This neuro-inflammatory response, besides promoting local vascular leakage through the blood-brain barrier (Zlokovic B, “Neurovascular pathways to neurodegeneration in Alzheimer's disease and other disorders,” Nature Reviews Neurosci. (2011) 12:723-738), has been implicated in driving further production of aberrant Aβ peptides 40/42 via modulation of gamma-secretase activity (Yan et al., “Anti-Inflammatory Drug Therapy Alters β-Amyloid Processing and Deposition in an Animal Model of Alzheimer's Disease,” J. Neurosci. (2003) 23:7504-7509; Karran, 2011) and to be detrimental to hippocampal neurogenesis in the adult brain. Gasparini, et al., “Non-steroidal anti-inflammatory drugs (NSAIDs) in Alzheimer's disease: old and new mechanisms of action,” J. Neurochem (2004) 91:521-536. Thus, neuro-inflammation, in combination with amyloid oligomer-mediated neuronal intoxication, creates a cycle that results in progressive neural dysfunction and neuronal cell death spreading throughout the brain in subjects with AD.

Researchers believe that future treatments to slow or stop the progression of AD and preserve brain function (disease-modifying treatments) will be most effective when administered during the early stages of the disease. In the future, biomarker imaging will be essential to identifying which individuals are in these early stages and should receive disease-modifying treatment when it becomes available. Imaging technology will also be critical for monitoring the effects of treatment and tailoring a course of action.

As mentioned above, the accumulation of Aβ neuritic plaques along with neurofibrillary tangles containing hyperphosphorylated tau protein, are considered the neuropathological hallmarks of AD. In recent years, intensive research has indicated that the relative levels of Aβ and phosphorylated tau in the cerebrospinal fluid (CSF) can effectively be used as biomarkers to predict the presence of AD neuropathology. Blennow K., “Biomarkers in Alzheimer's disease drug development,” Nat Med. (2010) 16:1218-22. More specifically, studies have shown that the CSF levels of Aβ are significantly decreased while the CSF levels of phosphorylated tau are significantly increased in AD patients as well as in MCI patients who later convert to AD when compared to healthy control patients. Andreasen, et al. “Sensitivity, specificity, and stability of CSF-tau in AD in a community-based patient sample,” Neurology. (1999) 53:1488-94; Buchhave et al., “Cerebrospinal fluid levels of β-amyloid 1-42, but not of tau, are fully changed already 5 to 10 years before the onset of Alzheimer dementia,” Arch Gen Psychiatry. (2012) 69:98-106; Lanari, et al., “Cerebrospinal fluid biomarkers and prediction of conversion in patients with mild cognitive impairment: 4-year follow-up in a routine clinical setting,” Scientific World Journal. (2009) 9:961-6; Monge-Argilés et al. “Biomarkers of Alzheimer's disease in the cerebrospinal fluid of Spanish patients with mild cognitive impairment,” Neurochem Res. (2011) 36:986-93; and Sunderland et al., “Decreased beta-amyloid1-42 and increased tau levels in cerebrospinal fluid of patients with Alzheimer disease,” JAMA. (2003) 289:2094-103.

Importantly, relative changes in these biomarkers can be seen years before the manifestation of Alzheimer's dementia. Buchhave, 2012. In fact, in a study of 137 MCI patients, Buchhave et al. demonstrated that 90% of MCI patients who displayed pathological biomarker levels at baseline developed AD within 9 to 10 years, and that the CSF levels of Aβ were fully decreased at least 5 to 10 years before the conversion to AD dementia. Id. In an analysis of 203 patients (131 with AD and 72 controls), Sunderland et al suggested that thresholds of 444 pg/mL for CSF Aβ and 195 pg/mL for CSF tau gave a sensitivity and specificity of 92% and 89%, respectively, to distinguish AD patients from controls. Sunderland, 2003. Similarly, Andreasen et al. found that a cutoff of 302 pg/mL for CSF tau resulted in a sensitivity and specificity of 93% and 86%, respectively, for distinguishing AD patients from control patients. Andreasen, 1999.

### SUMMARY OF THE INVENTION

The invention encompasses methods of treating Alzheimer's Disease comprising administering to a subject in need thereof a therapeutically effective amount of cromolyn. One embodiment encompasses wherein the cromolyn is cromolyn sodium. The method may further comprise administering ibuprofen. Another embodiment includes where cromolyn is administered to 17.1 mg. Yet another embodiment encompasses wherein ibuprofen is administered in an amount of 10 mg. One embodiment includes where cromolyn is delivered orally, via inhaler, intravenously, intraperitoneally, or transdermally. Another embodiment includes where the therapeutically effective amount of cromolyn decreased Aβ by about 10 to 50% after one week of treatment.

The invention encompasses methods where the cromolyn is administered to achieve a cromolyn concentration in plasma of about 14-133 ng/ml. An embodiment includes where the cromolyn is administered to achieve a cromolyn concentration in plasma of about 46 ng/ml. Another embodiment includes wherein the cromolyn concentration in plasma is achieved at about 6-60 minutes. Yet another embodiment includes wherein the cromolyn concentration in plasma is achieved in about 22 minutes.

The invention also encompasses methods wherein the cromolyn achieves an average Cmax cromolyn concentration in the CSF of about 0.3 to about 0.4 ng/ml. An embodiment includes wherein the cromolyn achieves an average Cmax cromolyn concentration in the CSF of about 0.24 ng/ml. Another embodiment includes methods wherein the ibuprofen achieves an average Cmax in the CSF of about 2.3 to 5.2 g/nl. Yet another embodiment includes methods wherein the ibuprofen achieves an average Cmax in the CSF of about 3.94 g/nl. An embodiment includes methods wherein the ibuprofen Cmax is achieved in about 2-4 hours. Another embodiment includes methods wherein the ibuprofen Cmax is achieved in about 2.55 hours. Yet another embodiment includes methods wherein the ibuprofen achieves an average Cmax ibuprofen concentration in plasma of about 25 to about 1970 ng/ml. Another embodiment includes methods wherein the ibuprofen achieves an average Cmax ibuprofen concentration in plasma of about 1091 ng/ml.

### BRIEF DESCRIPTION OF THE DRAWINGS

FIGS. 1A-D. FIG. 1A illustrates the chemical structures for cromolyn sodium and fesitin. FIG. 1B illustrates the effect of cromolyn sodium on Aβ₄₀ and Aβ₄₂ fibrillization was tested over one hour of incubation at 37° C. with increasing concentrations of cromolyn sodium (5, 50, 5000 nM) inhibited Aβ fibril formation in vitro at a nanomolar concentration. FIG. 1C illustrates cromolyn sodium inhibition of Aβ polymerization in vitro, using TEM, the formation of Aβ₄₂ fibrils was inhibited after incubation with 500 nM of cromolyn sodium. FIG. 1D illustrates treatment of HEK293 cells overexpressing both N- or C-terminal of luciferase conjugated Aβ₄₂ with cromolyn sodium that significantly decreased the luminescence signal in a dose-dependent manner FIG. 1E illustrates the effect of cromolyn sodium to conditioned media that already contained pre-existing oligomers and failed to impact the luminescence signal.

FIGS. 2A-C. FIG. 2A illustrate Aβ aggregation after acute exposure of AD transgenic mice with 2.1 mg/kg or 3.15 mg/kg cromolyn sodium for seven days significantly lowered the content of both TBS-soluble Aβₓ₋₄₀ and Aβₓ₋₄₂ by more than 50% (2.1 mg/kg dose: 39.5% for Aβₓ₋₄₀, 40.9% for Aβₓ₋₄₂; 3.15 mg/kg dose: 37.1% for Aβₓ₋₄₀ 46.2% for Aβₓ₋₄₂ respectively). FIG. 2B illustrates the concentrations of Aβ oligomers measured using the 82E1/82E1 ELISA assay noting that no changes in the levels of oligomeric aggregates could be detected. FIG. 2C illustrates quantification of the 4 kDa Aβ band using 6E10 and 82E1 detection antibodies that showed that cromolyn sodium decreased the amounts of monomeric Aβ.

FIGS. 3A-B. FIG. 3A illustrates concentrations of Aβ detergent resistant species sequentially extracted in 2% triton. FIG. 3B illustrates concentrations of Aβ detergent resistant species sequentially extracted in 2% SDS (FIG. 3B).

FIGS. 4A-D. FIG. 4A illustrates the impact of cromolyn sodium on the most insoluble fraction of Aβ peptides (formic acid extracts) and on the density of amyloid deposits. FIG. 4B illustrates that cromolyn sodium only impacted the soluble pool of Aβₓ₋₄₀ and Aβₓ₋₄₂ in TBS, Triton and SDS extracts, and it did not overall alter the distribution of Aβ peptides within each biochemical fraction (TBS, Triton, SDS, and formic acid). FIGS. 4C and 4D illustrate the quantification of the amyloid burden and the density of amyloid deposits, assessed immunohistochemically with an anti-Aβ antibody, confirmed that the amount of extracellular deposited aggregates of amyloid peptides remained unaffected after one week of cromolyn sodium treatment.

FIGS. 5A-B. FIG. 5A illustrates that administration of cromolyn sodium decreased ISF Aβₓ₋₄₀ level by 30% (PBS: 387 pM, cromolyn 283 pM). FIG. 5B illustrates that both ISF Aβₓ₋₄₂ and Aβ oligomers performed similarly in the test.

FIGS. 6A-B. FIG. 6A illustrates that in mice injected with cromolyn sodium ISF Aβ levels started to decrease only 2 hours after administration of Compound E, significantly faster than in PBS treated mice. FIG. 6B illustrates that the half-life of ISF Aβ in cromolyn sodium treated mice was shorter than control by about 50%.

### DETAILED DESCRIPTION OF THE PRESENT INVENTION

The invention encompasses methods of treating Alzheimer's disease (AD) by the administration of low doses of cromolyn to a subject in need thereof, wherein the lose dose inhibits aggregation of Aβ monomers into higher order oligomers and fibrils. The methods may further comprise the administration of ibuprofen either simultaneously or sequentially with cromolyn to treat AD. The invention also comprises methods of treating AD by administering cromolyn to a subject in need thereof in a sufficient amount to decrease soluble levels of Aβ about 10% to about 50% after at least one week of treatment. Not to be limited by theory, it is believed that a method of treating AD is based on inhibiting the aggregation of Aβ monomers into higher order oligomers and fibrils in vitro, without affecting Aβ production. Misfolded Aβ monomers can aggregate into higher order oligomers, eventually forming fibrils that get deposited into the extracellular space to form fibrillary amyloid neuritic plaques. Aβ oligomers rather than monomers have been shown to be neurotoxic for neurons, inhibiting LTP, leading to neuronal stress, abnormal tau phosphorylation, synapse collapse, and memory impairment. Therefore, therapeutic agents that are able to decrease Aβ levels, prevent oligomer formation, or disaggregate soluble oligomers may be of therapeutic interest.

A low dose oral anti-inflammatory is theorized to inhibit the neuro-inflammatory response in persons with early AD. The cascade of Aβ oligomer-mediated neuronal intoxication is exacerbated by another AD trigger: chronic local inflammatory responses in the brain. Krstic, 2012. AD has a chronic neuro-inflammatory component that is characterized by the presence of abundant microglial cells associated with amyloid plaque. Heneka, 2005, and Imbimbo, 2010. These cyclooxygenase (COX1/COX2)-expressing microglia, which phagocytose amyloid oligomers, then become activated to secrete pro-inflammatory cytokines. Hoozemans, 2011; Griffin, 2011; and Krstic, 2012. This neuro-inflammatory response, besides promoting local vascular leakage through the blood-brain barrier (Zlokovic, 2011), has been implicated in driving further production of aberrant Aβ peptides 40/42 via modulation of gamma-secretase activity (Yan, 2003; Karran, 2011) and in inhibiting, hippocampal neurogenesis in the adult brain (Gasparini, 2004). Thus, neuro-inflammation, in combination with amyloid oligomer-mediated neuronal intoxication, creates a cycle that results in progressive neural dysfunction and neuronal cell death spreading throughout the brain in subjects with AD.

Compelling evidence from multiple epidemiology studies revealed that long-term dosing with non-steroidal anti-inflammatory drugs (NSAIDs) dramatically reduced AD risk in the elderly, including delayed disease onset, reduced symptomatic severity and slowed cognitive decline. Veld et al., “Nonsteroidal Antiinflammatory Drugs and the Risk of Alzheimer's Disease,” N. Engl. J. Med (2001) 345:1515-1521; Etminan et al., “Effect of non-steroidal anti-inflammatory drugs on risk of Alzheimer's disease: systematic review and meta-analysis of observational studies,” Brit. Med. Journal (2003) 327:1-5; Imbimbo, 2010). Three mechanisms have been proposed to explain how NSAIDs inhibit the processes that contribute to AD progression:

a) by inhibiting COX activity, thereby reducing or preventing microglial activation and cytokine production in the brain (Mackenzie et al., “Nonsteroidal anti-inflammatory drug use and Alzheimer-type pathology in aging,” Neurology (1998) 50:986-990; Alafuzoff et al., “Lower counts of Astroglia and Activated Microglia in Patients with Alzheimer's Disease with Regular Use of Non-Steroidal Anti-inflammatory Drugs,” J. Alz. Dis. (2000) 2, 37-46; Yan, 2003; Gasparini, 2004; Imbimbo, 2010);

b) by reducing amyloid deposition (Weggen et al., “A subset of NSAIDs lower amyloidogenic 442 independently of cyclooxygenase activity,” Nature (2001) 414:212-216; Yan, 2003; Imbimbo, 2010);

c) by blocking COX-mediated prostaglandin E2 responses in synapses. Kotilinek, et al., “Cyclooxygenase-2 inhibition improves amyloid-β-mediated suppression of memory and synaptic plasticity,” Brain (2008) 131:651-664.

Dampening the neuro-inflammatory response will impact AD progression by several mechanisms. Ibuprofen, which crosses the human blood brain barrier (Bannwarth B., “Stereoselective disposition of ibuprofen enantiomers in human cerebrospinal fluid,” Br. J. Clin. Pharmacol. (1995) 40:266-269; Parepally, et al., “Brain Uptake of Nonsteroidal Anti-Inflammatory Drugs: Ibuprofen, Flurbiprofen, and Indomethacin,” Pharm. Research (2006) 23:873-881), dampens the production of pro-inflammatory cytokines (Gasparini, 2004), which should contribute to its utility for preventing AD progression. However, when NSAIDS such as rofecoxib and naproxen have been administered as monotherapy in clinical trials for the treatment of AD, the results have either been inconclusive or have indicated a higher risk of AD progression when administered as the sole therapy in clinical trials (Thal, et al., “A Randomized, Double-Blind, Study of Rofecoxib in Patients with Mild Cognitive Impairment,” Neuropsychopharmacology (2005) 30:1204-1215; Imbimbo, 2010) despite the multiple epidemiology studies showing reduced AD risk in individuals taking NSAIDs, including ibuprofen (Veld, 2001; Etminan, 2003). Besides the criticism surrounding the choice of NSAIDs, such as rofecoxib and naproxen for monotherapy in AD (Gasparini, 2004), the ADAPT rofecoxib/naproxen treatment trial was conducted with subjects exhibiting mild-to-moderate AD. Aisen et al., “Effects of Rofecoxib or Naproxen vs. Placebo on Alzheimer Disease Progression,” JAMA (2003) 289:2819-2826; Breitner et al., “Extended results of the Alzheimer's disease anti-inflammatory prevention trial,” Alz. Dementia (2011) 402-411. Given the epidemiology data, it has been hypothesized that NSAID administration may be beneficial only in the very early stages of the disease. Imbimbo, 2010; Breitner, 2011. Thus, this study has been designed to specifically target patients with clinical evidence of early AD.

It is also important to note that in the NSAID epidemiology studies, reduced risk of AD was restricted to NSAIDs that presumably lowered Aβ-42 peptide levels, such as ibuprofen and indomethacin (Gasparini, 2004; Imbimbo, 2010). Also worth noting is that long-term dosing with low NSAID doses are as equally effective as higher doses. Breitner J., “Alzheimer's disease: the changing view,” Annals Neurol. (2001) 49:418-419; Broe et al., “Anti-inflammatory drugs protect against Alzheimer's disease at low doses,” Arch Neurol. (2000) 57:1586-1591.

The inflammatory response has been correlated with amyloid production and oligomeric low concentration. Therefore, the ibuprofen dose of the invention is calculated to treat at least that amount, while minimally affecting the systemic toxicity.

Ibuprofen is approved for pain and as described above is used to treat inflammation. For moderate to strong pain and inflammation physicians subscribe up to 800 mg dose 4 times a day (3200 mg). This dose could be given for a maximum of two weeks. The total treatment dose for this treatment is 3200 mg/day×14 days is 44,800 mg equal to 217 mM. Continued use of this daily dosing is associated with severe side effects. The over the counter dose is 200 mg. Some may use multiple doses per day and others may use one daily.

The yearly consumption of one dose a day totals 73,000 mg per year. The proposed dose for treating the “invisible” neuro-inflammatory response for the estimated daily abeta that converts to Amyloid plaque (22-27 ng/day) (reference) could be achieved by administering 10 mg/day, which is equal to 3650 mg/year. This yearly dose is 13 times less than the two week maximum dose or 20 times less than over the counter yearly dose for pain. The advantage of the proposed dose is the elimination of the chronic use of the drug.

The dose rationale and calculation for ALZT-OP1b (ibuprofen) are as follows:

(RS)-2-(4-(2-methylpropyl)phenyl) propanoic acid) MW=206 Da (206 g/mol)

The oral absorption into plasma is 98%. The brain uptake from protein bound ibuprofen=5% of total and the free ibuprofen concentration in plasma=0.5% of total plasma ibuprofen. Therefore, 5.5% of dose in plasma, with a range from 1-4% brain uptake from plasma. For example: 10 mg ibuprofen×98%=9.8 mg ibuprofen in plasma following absorption from oral tablet and 9.8 mg×5.5% available for brain uptake=0.54 mg, therefore, range of uptake is 1-4% dose in plasma=5.4 ee-4 g×1% brain uptake=5.4 ee-6 g/206 g/mol=2.6 ee-8 mol/1.5 L brain volume=17.5 nM ibuprofen per L brain. The calculation for 4% was as follows: 21.6 ee-3 g×4% brain uptake=21.6 ee-6 g/206 g/mol=1.05 ee-7 mol/1.5 L brain volume=70 nM (or four times the 1%) per L brain. Therefore, 10 mg ibuprofen tablet was estimated to result in 17.5-70 nM concentration in the brain. This concentration correlated, as gross estimate, to treat the potential inflammatory response triggered by the Aβ daily production.

The evaluation of the plasma and CSF levels in 24 human subjects under an IND and a phase I study followed a 10 mg or 20 mg oral administration to healthy volunteers (age 55-79).

Preliminary PK profile of ibuprofen in plasma was characterized by an irregular absorption pattern, often with a lag time. The human pharmacokinetics data show that ibuprofen concentration in plasma for 10 mg oral administration resulted in a Cₘₐₓ 1091±474.6 ng/ml (range: 25.5-1970.0 ng/ml) at 95.4±85.9 min (range 12 min to 6 h). The apparent t_{1/2} in plasma was 1.93±0.32 h (range 1.5 to 2.5 h) indicating moderate clearance from plasma.

The average Cₘₐₓ of ibuprofen in the CSF during the observed time interval of up to 4 hours was 3.94±1.292 ng/ml (range 2.3 to 5.2 ng/ml) at 2.55±0.961 h (range 2.0 to 4.0 h) following oral administration of a 10 mg dose. It was estimated that this level of ibuprofen in the brain (19.2±6.3 nM) was sufficient to treat the potential inflammatory response caused by the Aβ daily production.

Therefore, 10 mg ibuprofen tablet is estimated to result in brain concentrations (836 ng) or larger 4 times larger than the required dose to treat 22-27 ng. This nanomolar ibuprofen brain concentration is estimated to treat the potential inflammatory response caused by the Aβ daily production. In some embodiments this drug dose is combined as mixture with one or more anti-amyloid drugs as one specific treatment or as an adjuvant to the standard disease treatment.

In summary, NSAIDs are predicted to dampen the neuro-inflammatory response and impact AD progression via several mechanisms. When administered together with drugs that inhibit AP oligomerization.

To determine the cromolyn dose example we calculated as follows. Sodium cromoglycate: 5,5′-(2-hydroxypropane-1,3-diyl)bis(oxy)bis(4-oxo-4H-chromene-2-carboxylic acid) MW=512 Da (512 g/mol). The dose rationale and calculation for cromolyn was as follows. (1) Dry powder inhaler (DPI) results show 4-5 mg cromolyn (in the impactor fractions with <3 μm size particles needed for systemic uptake) per 17.1 mg of API, to be delivered to the lower respiratory tract for systemic uptake. 4-5 ee-3 g/512 g/mol=7.8-9.8 micromoles of cromolyn plasma levels. If cromolyn was 0.2-1% uptake in brain from plasma=16-98 nanomoles divided by/1.5 L brain=11-66 nM cromolyn/L in brain (per day). Therefore, 17.1 mg cromolyn inhaled with AZHALER device was estimated to result in 11-66 nM concentration in the brain.

The human pharmacokinetics data show that cromolyn concentration in plasma reached maximum of 46.7±33.0 ng/ml (range: 14-133 ng/ml) at 22.8±16.6 min (range: 6-60 min) upon inhalation of 17.1 mg dose of cromolyn. Cromolyn clearance from plasma was rapid, with a half-life of 1.75±0.9 h (range: 0.6-3.7 h). The average Cₘₐₓ cromolyn concentration in the CSF following 17.1 mg cromolyn inhalation was 0.24±0.077 ng/ml (range: 0.2-0.4 ng/ml) at 3.72±0.704 h, corresponding to 0.47±0.15 nmol/L. It was estimated that this level of cromolyn in the brain (0.47 nmol/L×1.5 L=0.70 nmol), was sufficient to titrate the estimated daily 22-27 ng (27 ng/512 MW=0.06 nmol) of amyloid plaque and the associated inflammatory response.

And 34.2 mg dose inhalation was in the range 0.36±0.17 ng/ml (range: 0.16-0.61 ng/ml), corresponding to cromolyn concentration of 0.71 nM. Assuming the 4 hours is the maximum with a similar washout profile for 8 hours, will extrapolates to a CSF doubled concentration of 1.41 nM. This concentration translates to more than one order of magnitude (23 times) higher than the amount to titrate the estimated 22-27 ngr (27 ngr/512 MW=0.06 nM) plaque produced in the brain per day. This, 17.1 mg, proposed chronic daily dose is sufficient to slowdown or holt the polymerization without affecting potential long run toxicity use of the drug.

In some embodiments cromolyn and other anti Aβ agents in the specified doses or calculated doses to titrate disease progression as separate treatment or as combination (separately delivered ore as mixture) with other neurodegenerative targeted disease, such as Alzheimer's are proposed.

The combination treatment paradigm is proposed to attenuate the multiple triggers leading to neurodegeneration and neuronal death. This decline in cognitive performance may be reversed, due to preserved or improved neuronal plasticity and neurogenesis in the hippocampus (Kohman, et al., “Neurogenesis, inflammation and behavior,” Brain, Behavior, and Immunity (2013) 27:22-32), if AD progression is arrested at a very early stage. The combination treatment paradigm is proposed to improve cognition and function as an adjuvant addition to standard treatment to optimize outcome.

The mitigation of AD progression could potentially improve quality of life for patients in addition to ameliorating the expensive health care costs in the long term care of patients with progressive AD.

The investigational product ALZT-OP1b (ibuprofen) is non-selective COX inhibitor for treating inflammation as an NSAID. Other members of this class include aspirin, celecoxib, diclofenac, ketoprofen, ketorolac, naproxen, piroxicam and sulindac. These drugs are commonly used for the management of mild to moderate pain, fever, and inflammation and also has an antiplatelet effect, though less than aspirin.

The COX enzymes convert certain fatty acids to prostaglandins. Ibuprofen, taken in accordance with drug labeling, works by blocking the production of prostaglandins, substances our body releases in response to illness and injury. Prostaglandins cause pain and swelling (inflammation); they are released in the brain and can also cause fever. The prostaglandins at the end of the “chain” of reactions that starts with the COX enzyme cause an increased sensitivity to pain, fever, and vasodilation (increased blood flow or inflammation). By inhibiting the start of this chain of reactions, ibuprofen therefore reduces pain, fever, and inflammation. Because ibuprofen blocks the activity of both COX enzymes, it is considered a non-selective COX inhibitor NSAID.

As described above, dampening the neuro-inflammatory response will impact AD progression by several mechanisms. Ibuprofen, which crosses the human blood brain barrier (Bannwarth, 1995; Parepally, 2006), dampens the production of pro-inflammatory cytokines (Gasparini, 2004), which should contribute to its utility for preventing AD progression. However, when NSAIDs such as rofecoxib and naproxen have been administered as monotherapy in clinical trials for the treatment of AD, the results have either been inconclusive or have indicated a higher risk of AD progression (Thal, 2005; Imbimbo, 2010), despite multiple epidemiology studies showing reduced AD risk in individuals taking NSAIDs, including ibuprofen (Veld, 2001; Etminan, 2003). Besides the criticism surrounding the choice of NSAIDs such as rofecoxib and naproxen for monotherapy in AD (Gasparini, 2004), the ADAPT rofecoxib/naproxen treatment trial was conducted with subjects exhibiting mild-to-moderate AD (Aisen 2003; Breitner, 2011). Given the epidemiology data, it has been hypothesized that NSAID administration may be beneficial only very early in disease (Imbimbo, 2010; Breitner, 2011). Thus, patients presenting with clinical evidence of early AD have been selected for study in this clinical trial.

It is important to note that in the NSAID epidemiology studies, AD risk decrease was restricted to NSAIDs that presumably lowered Aβ-42 peptide levels, such as ibuprofen and indomethacin (Gasparini, 2004; Imbimbo, 2010), and long-term dosing with low NSAID doses were as equally effective as higher doses (Broe, 2000; Breitner 2001). Hence, in one cohort in this AZTherapies ALZT-OP1 trial, 10 mg ibuprofen will be administered as oral tablets (ALZT-OP1b). This dose is significantly lower than the over-the-counter approved dose. In combination with cromolyn inhalation treatment (ALZT-OP1a), we will test the hypothesis that dampening the low level neuro-inflammatory response with ibuprofen will contribute significantly to preventing cognitive decline due to AD progression.

Ibuprofen (ALZT-OP1b) belongs to the class of non-steroidal anti-inflammatory drugs (NSAIDs). For this study, a 10 mg ibuprofen tablet will be taken daily (orally) at the same time each day as ALZT-OP1a for prevention and or slowing the effect neuro-inflammatory response seen in AD. This drug is FDA-approved and has been available for many years over-the-counter (OTC), however, a smaller dose than available OTC will be used for this study.

The active ingredient of ibuprofen tablets, USP is (±)-2-(p-isobutylphenyl) propionic acid, making it an organic compound in the class of propionic acid derivatives. Ibuprofen is a stable white crystalline powder with a melting point of 74-77° C. and is very slightly soluble in water (<1 mg/mL) and readily soluble in organic solvents such as ethanol and acetone. It's pKa is 4.4-5.2.

| | Compendial | | | |
|---|---|---|---|---|
| Component | Status | Function | % w/w | mg/tab |
| Ibuprofen | USP/NF | Active Pharmaceutical | 10.0 | 10.0 |
| | | Ingredient | | |
| Mannitol | USP/NF | Filler | 59.5 | 59.5 |
| (Pearlitol 100SD) | | | | |
| Microcrystalline cellulose | USP/NF | Filler | 25.0 | 25.0 |
| (Avicel PH102) | | | | |
| Croscarmellose sodium | USP/NF | Disintegrant | 4.0 | 4.0 |
| (Solutab type A) | | | | |
| Magnesium stearate | USP/NF | Lubricant | 1.5 | 1.5 |
| (Ligamed MF-2-V) | | | | |
| Sub-total | | | 100 | 100 |
| Opadry ® 20A19301 clear | House | Protective sub-coating | 2.0 | 2.0 |
| Acryl-EZE ® MP 93O18508 | House | Enteric coating | 5.0 | 5.0 |
| white | | | | |

| | |
|---|---|
| Total | 107 |

Route of Administration, Dosage, Regimen, and Treatment Period

Ibuprofen may be taken once daily by mouth (orally) with water for the duration of treatment.

Tablets may be enterically coated to control the location in the digestive system where the drug will be absorbed in order to avoid possible undesirable side effects such as gastrointestinal ulcers and stomach bleeding associated with chronic dosing of NSAID' s. The enterically coated tablet is intended to bypass the highly acidic environment in the stomach (approx. pH 3) and dissolve in a more basic environment (approx. pH 7-9) found in the small intestine. The daily dose of ibuprofen for this embodiment is 80-100 times less than prescribed daily dose for pain, fever, and inflammation.

Description of Cromolyn

The investigational product ALZT-OP1a (cromolyn) is a synthetic chromone derivative that has been approved for use by the FDA since the 1970s for the treatment of asthma. For asthma treatment, cromolyn powder was micronized for inhalation to the lungs via dry powder inhaler, the Spinhaler device. Liquid intranasal and ophthalmic formulations have also been developed for the treatment of rhinitis and conjunctivitis.

The mechanism of action for cromolyn is characterized as a mast cell stabilizer, namely to suppress cytokine release from activated lymphocytes together with preventing the release of histamine from mast cells (Netzer et al., “The actual role of sodium cromoglycate in the treatment of asthma—a critical review,” Sleep Breath (2012) 16:1027-1032; Keller, et al., “Have inadequate delivery systems hampered the clinical success of inhaled disodium cromoglycate?” Time for reconsideration. (2011) 8:1-17. It was administered four times daily as prophylaxis for allergic and exercise-induced asthma, not as a treatment for acute attacks.

Our studies have shown a new mechanism of action for cromolyn, which, along with its role for suppressing immune responses, enables the re-purposing of this approved drug for use to potentially halt or slow AD progression. These studies have shown that cromolyn binds to AP peptides and inhibits its polymerization into oligomers and higher order aggregates. The inhibition of Aβ polymerization will arrest amyloid-mediated intoxication of neurons and restore the passage of these aberrant Aβ oligomers out of the brain rather than their accumulation. Furthermore, we have shown that cromolyn penetrates the blood-brain barrier in animal models, so that plasma bioavailability following cromolyn inhalation will translate to concentrations in the brain sufficient to interfere with Aβ oligomerization and accumulation.

Our studies with an Aβ animal model using APP/PS1 transgenic mice (which develop amyloid burden in the brain) provided statistically significant evidence of the benefit with ALZT-OP1a treatment. Administration of cromolyn, but not mock treatment, to the transgenic animals prevented lowering memory capabilities in the Morris water maze tests seen with age-matched healthy non-transgenic animals. Similar administration of two other known amyloid-binding agents failed to provide any benefit in this Alzheimer transgenic animal model. These results indicate that ALZT-OP1a treatment slowed down the decline in learning and memory caused by brain amyloid burden in a transgenic animal model of AD.

Cromolyn sodium is the disodium salt of 5,5′-[(2-hydroxytrimethylene)dioxy]bis [4-oxo-4H-1-benzopyran-2-carboxylate] and is a water soluble, odorless, white, hydrated crystalline powder.

TABLE 1
ALZT-OP1a (cromolyn) Formulation

| | | | ALZT-OP1a Composition | | | |
|---|---|---|---|---|---|---|
| | | | Placebo | | Drug Product | |
| | Quality | | % | mg/ | % | mg/ |
| Component | Standard | Function | w/w | capsule | w/w | capsule |
| Cromolyn sodium | USP | Active | — | — | 58.0 | 17.1^{a} |
| (micronized) | | | | | | |
| Lactose monohydrate | NF | Diluent | 98.0 | 44.1 | 40.0 | 12.8 |
| Magnesium stearate | NF | Stabilizer | 2.0 | 0.9 | 2.0 | 0.6 |
| (micronized) | | | | | | |
| Hydroxypropyl | In-house | Encapsulation | NA | NA | NA | NA |
| methylcellulose capsule^{b} | | | | | | |

| | | | | |
|---|---|---|---|---|
| Total | 100% | 45 | 100% | 32 |

^{a}Weight of cromolyn sodium, USP per capsules is 17.1 mg on an anhydrous basis (18.6 mg per capsule on as-is basis).
^{b}Hydroxypropyl methylcellulose capsule functions only to meter and deliver the drug product through the dry powder inhaler and is not ingested during administration.

The amount of cromolyn in dose will depend on a variety of conditions of the subject, such as condition of the disease, health, age, sex, weight, among others. When the formulation is formulated for inhalation, typically, the amount of cromolyn in a single dose is about 5 to about 20 mg, preferably about 10 to 19 mg, and more preferably, the amount is about 15 to 18 mg. In one particular embodiment, that amount of cromolyn is about 17.1 mg.

For example, a formulation may contain cromolyn powder blend prepared for use with a dry powder inhaler device. Each unit will comprise 17.1 mg of the cromolyn and pharmaceutically acceptable excipients. The formulation may be administered twice daily (34.2 mg) that is less than 50% of the cromolyn dose from the four times daily approved dose level (80 mg cromolyn total per day) currently administered for the treatment of asthma.

For daily administration, typically, the amount of cromolyn would be about 5 mg to about 45 mg; preferably, the amount of the daily dose would be about 20 mg to about 38 mg, and more preferably, the amount would be about 30 gm to about 36 mg. For example, a daily dose of 34.2 mg cromolyn (17.1 mg cromolyn, inhaled twice daily, morning and evening using dry powder inhaler) would inhibit post stroke neuro-inflammation and limit mast cells migration/degranulation, glial activation, and neuronal loss and potentially slow down cognitive decline.

When administered with a ibuprofen, typically, the cromolyn is administered in an amount of about 17.1 mg and ibuprofen is administered in 20 mg (such as two orally administered 10 mg doses taken consecutively). Alternatively, cromolyn is administered in 34.2 mg (such as administration of two consecutive inhaled doses of 17.1 mg) and 20 mg of ibuprofen.

The manufactured capsules are blistered and packaged to prevent exposure to moisture, light, and other environmental factors that could negatively impact drug stability. All product packaging and labeling will be in accordance with cGMP, GCP, local, federal, and country specific regulations and requirements.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### EXAMPLES

Cromolyn sodium U.S.P. grade was purchased from Spectrum Chemical Mfg. Corp. (Gardena, Calif.) and dissolved in sterile phosphate buffered saline (PBS). A stock solution of 100 mM was used for in vitro experiments and 10.2 mM was used for in vivo administration. In vitro, cromolyn sodium stock solution was directly diluted in the cell culture media at final concentrations of 10 nM, 10 μM or 1 mM, while a solution of 1.02 mM of the compound was prepared in Bulbecco's Phosphate Buffer saline (DPBS) before intraperitoneal injection in vivo (at three different doses: 1.05 mg/kg, 2.1 mg/kg, or 3.15 mg/kg body weight). In vitro amyloid fibrillization assay was performed using synthetic Aβ peptides (rPeptide, Bogart GA) as well as thioflavin-T (Sigma-Aldrich), respectively dissolved in DMSO and in methanol. For the in vitro efflux and microglial uptake assay, synthetic Aβ₄₀ and Aβ₄₂ peptides were purchased from Peptide Institute, Inc. After resuspension in 1,1,1,3,3,3-hexafluoro-2-propanol (HFIP, Kanto Chemical) at a concentration of 1 mg/ml, the peptides were dried, resolubilized in PBS containing 2% (v/v) Me₂So (Kanto Chemical) and filtered through a 0.2 mm filter. The stock solution of Aβ₄₀ and Aβ₄₂ were applied at 50 nM in cell cultures.

### Example 1: In Vitro Aβ Fibrillization Oligomerization and Dissociation Assays

In vivo fibrillization assay was performed using Aβ₄₀ and Aβ₄₂ dissolved in DMSO at a concentration of 250 μM and sonicated for 1 min. Aβ₄₀ and Aβ₄₂ were diluted to 5 μM in an assay volume of 200 μl with artificial CSF solution (125 mM NaCl, 2.5 mM KCl, 1 mM MgCl₂, 1.25 mM NaH₂PO₄, 2 mM CaCl₂, 25 mM NaHCO₃, and 25 mM glucose, pH 7.3) in 96 well plate (Corning, Tewksbury, Mass.). After addition of 10 μM thioflavin-T and increasing concentrations of cromolyn sodium (5 nM, 50 nM, and 500 nM), the fibrillization process was initiated by adding 0.5 mg/ml of heparin sulfate (Sigma, St. Louis Mo.). DMSO was used as control. The progression of fibrillization was followed every 10 min for 60 min at room temperature by measuring the fluorescence intensity at excitation and emission wavelengths of 450 nm and 480 nm, respectively, using an M3 microplate reader. The results were normalized for background using fluorescent reading at time 0 by the software provided by the M3 plate reader.

Aβ agglomeration and oligomer dissociation assays were performed in vitro using an Aβ splitluciferase complementation assay. To evaluate the effect of cromolyn sodium on the formation of Aβ oligomers, a HEK293 cell line designed to stably overexpress the N- and C-terminal fragments of Gaussian luciferase (Gluc) conjugated to Aβ₄₂ was incubated without or with cromolyn sodium at 10 nM, 10 μM, or 1 mM for 12 hours at 37° C. The conditioned media from these cells was collected, 10 nM of coelenterazine was added and the luciferase activity was measured using a Wallac 1420 (PerkinElmer). The oligomer dissociation assay was performed by incubating PBS or cromolyn sodium (10 nM, 10 μM, or 1 mM) with conditioned media from naïve HEK293 cells overexpressing each half of Gluc fused with Aβ₄₂, 12 hours at 37° C. The luciferase activity was measured.

Analysis of Aβ₄₂ Fibril Formation by Transmission Electron Microscopy

The anti-fibrillogenic properties of cromolyn was confirmed by performing TEM analysis. Briefly, synthetic Aβ₄₂ was dissolved in PBS at a concentration of 0.2 mg/ml for 48 hours at 37° C., with or without addition of cromolyn sodium at a concentration of either 5 nM or 500 nM. After incubation for 48 hours, 15 μl or the Aβ₄₂ fibril solution were adsorbed on carbon-coated EM grids for 20 min at room temperature. After 3 washes in sterile PBS and ddH₂O, the grids were allowed to dry before negative staining with 2% (w/v) uranyl-acetate water, two times for 8 min Each grid was then briefly washed in degassed ddH₂O, air dried, and imaged by TEM at a magnification of 150,000×.

In Vitro Microglial Uptake Assay

In vitro evaluation of Aβ uptake was performed. Briefly, human microglial cells (HMG 030, Clonexpress, Inc., Gaithersburg, Md.) were isolated from fetal brain tissue samples and suspended in a culture medium (50:50 of DMEM: F-12) supplemented with 5% FBS, 1% penicillin/streptomycin, and 10 ng/mL of M-CSF. The isolated microglia cells were plated into glass-bottomed well plates and incubated at 37° C. supplied with 5% CO₂ for two days before treatment with Aβ and cromolyn sodium. After a medium change, microglia cells were incubated with 50 nM Aβ₄₂ with or without cromolyn sodium at 10 nM, 10 μM, or 1 mM for 16 hours at 37° C. After incubation, the medium was collected and the levels of Aβ₄₀ and Aβ₄₂ were measured using a two-site Aβ ELISA and microglial cells were fixed in 4% paraformaldehyde and the number counted.

Animals and Cromolyn Sodium Treatment

APPswe/PS1dE9 (APP/PSI) were purchased from the Jackson library. These mice express a human mutant K594N/M595 L as well as the Presenilin 1 gene deleted for the exon 9, both under the control of the prion promoter. This AD mouse model presents a severe phenotype with amyloid deposition beginning at 6 months of age. In the present study, 7.5 month-old APP/PS1 males were injected intraperitoneally (i.p.) daily for one week with escalating doses of 1.05 mg/kg, 2.1 mg/kg, or 3.15 mg/kg body weight of cromolyn sodium or PBS. For interstitial fluid (ISF) sampling, 9 month-old male APP/PS1 mice were i.p. injected daily with the highest dose of cromolyn sodium (3.15 mg/kg body weight) or PBS for 7 days, just before ISF sampling. One day after the last injection of ISF collection, the mice were euthanized by CO₂ inhalation. Plasma was then collected via cardiac puncture. After transcardiac PBS perfusion, the brain was dissected and one brain hemisphere was fixed in 4% paraformaldehyde for immunohistochemistry, whereas the contralateral hemisphere was snap-frozen in liquid nitrogen for biochemical assays.

Biochemical Sample Preparation

Brain tissue samples were homogenized in 10 volumes of TBSI (tris-buffered saline with protease inhibitor) with 25 strokes on a mechanical bouncer homogenizer and centrifuged at 260,000 g for 30 min at 4° C. The TBS soluble supernatant was collected and the pellet was then successively homogenized in 2% triton-100/TBSI, 2% SDS/TBSI and 70% formic acid.

Sandwich ELISA and Immunoblotting

The concentrations of Aβ₄₀ and Aβ₄₂ were determined using the commercially available kits BNT77/BA27 for Aβ₄₀ or BNT77/BC05 for Aβₓ₋₄₂, respectively. For guanidine (Gdn-HCl) treatment, samples were incubated with 0.5 M Gdn-HCl at 37° C. for 30 min Oligomeric Aβ species were quantified using the 82EI/82WI ELISA kit, in which both capture and detection antibodies are identical. For immunoblotting, TBS-soluble fractions were electrophoresed on a 10-20% Novex tris-glycine gels. After transfer on nitrocellulose membrane, the blots were blocked in 5% nonfat skim milk/TBST (tris-buffer saline with 0.1% Tween 20) buffer for 1 hour. Membranes were then probed with the anti-Aβ antibodies 6E10 and 82E1 overnight at 4° C. Following incubation with horseradish peroxidase-conjugated secondary antibody Mouse True Blot for 1 hour at room temperature, immunoreactive proteins were developed using an ECL kit and detected on Hyperfilm ECL. Aβ signal intensity was measured by densitometry using Image J software.

Immunochemistry

Serial paraffin sections were cut at 4-μm and immunostained with a rabbit anti-human amyloid (N) antibody for amyloid plaques, followed by biotinylated goat anti-rabbit secondary antibody and developed using the ABC Elite and DAB kits. Images were taken using an Olympus BX51 epifluorescence upright microscope equipped with a CCD camera model DP70. Quantitative analyses of amyloid load and plaque density were done using the BIOQUANT software after application of an optical threshold. This software is coordinated with the motorized stage of an upright Leica DMRB microscope equipped with a CCD camera. Immunostained amyloid plaques were thresholded under the 10×objective after background correction to avoid uneven lighting. For colocalization analysis of Aβ in microglia, 4-μm paraffin sections were immunostained with mouse anti-Aβ antibody 6E10 for Aβ and rabbit anti-Iba1 for microglia followed by Alexa 488- or Cy3-conjugated secondary antibodies. Images were acquired on a Zeiss LSM 510 META confocal microscope, using the same pinhole settings and gain for taking all the pictures between PBS and cromolyn sodium treated animals. The percentage of Iba1 colocalizing with amyloid deposits was determined after image analysis using the Fiji software. The exact same thresholds were applied to both 488 and Cy3 channels and an ROI was selected corresponding to each plaque. After application of this ROI on the Cy3 channel (Iba1 staining), an analysis of particles within the ROI was performed and the % of Iba1 staining overlapping with each amyloid deposit was measured.

In Vivo Microdialysis

In vivo microdialysis for ISF Aβ sampling was performed. Briefly, the mice were stereotactically implanted with two guide cannulas into both hippocampi (AP −3.1 mm, L+/−2.8 mm, DV −1.1 mm), under anesthesia with isoflurane (1.5% in O₂). After a recovery time of three days, i.p. injections of cromolyn sodium started. ISF sampling was done one week after exposure with cromolyn sodium or PBS as control. For ISF sampling, a 1000 kDa molecular probe was used. Before use, the probe was washed with artificial cerebrospinal fluid (aCSF: in mM: 122 NaCl, 1.3 CaCl₂, 1.2 MgCl₂, 3.0 KH₂PO₄, 25.0 NaHCO₃). The probe's outlet and inlet were then connected to a peristaltic pump and a microsyringe pump, respectively, using fluorinated ethylene propylene (FEP) tubing. The probe was inserted into mice hippocampus through the guide cannula. After implantation, aCSF was perfused for 1 hour at a flow rate of 10 μl/min before ISF sampling. ISF samples for the measurement of total Aβ or oligomeric Aβ were collected at a flow rate of 0.5 μl/min or 0.1 μl/min, respectively, and stored at −80° C. until Aβ measurement. During in vivo microdialysis sampling, mice were awake and freely moving in the microdialysis cage designed to allow unrestricted movement without applying pressure on the probe assembly.

Compound E Treatment Using Reverse Microdialysis

The contralateral hippocampus was used for this experiment. After baseline sampling for 4 hours, 100 mM of γ-secretase inhibitor Compound E diluted in aCSF was perfused into the hippocampus to rapidly inhibit Aβ production in the tissue surrounding the probe. Aβ levels within the ISF were measured for an additional 5 hours. The single logarithmic plot was made from Aβ levels and extrapolated the half-life of ISF Aβ.

Statistics Analysis

Statistical analyses were performed using Graph Pad 5 Prism software. In vitro, each experiment was performed at least three times independently and normality was verified. Comparison of means among three or more groups was analyzed using a one-way ANOVA followed by a Bonferroni's post hoc test. In vivo data were averaged per mouse and analyzed using a non-parametric Kruskal-Wallis test, followed by a Dunn's Multiple Comparison Test. For the quantification of amyloid plaques, data were analyzed using a non-parametric Mann-Whitney test. P values less than 0.05 were considered significant.

Results

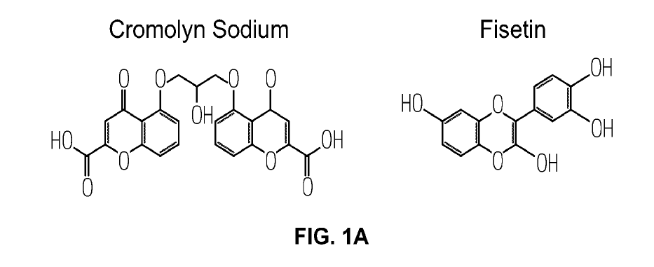

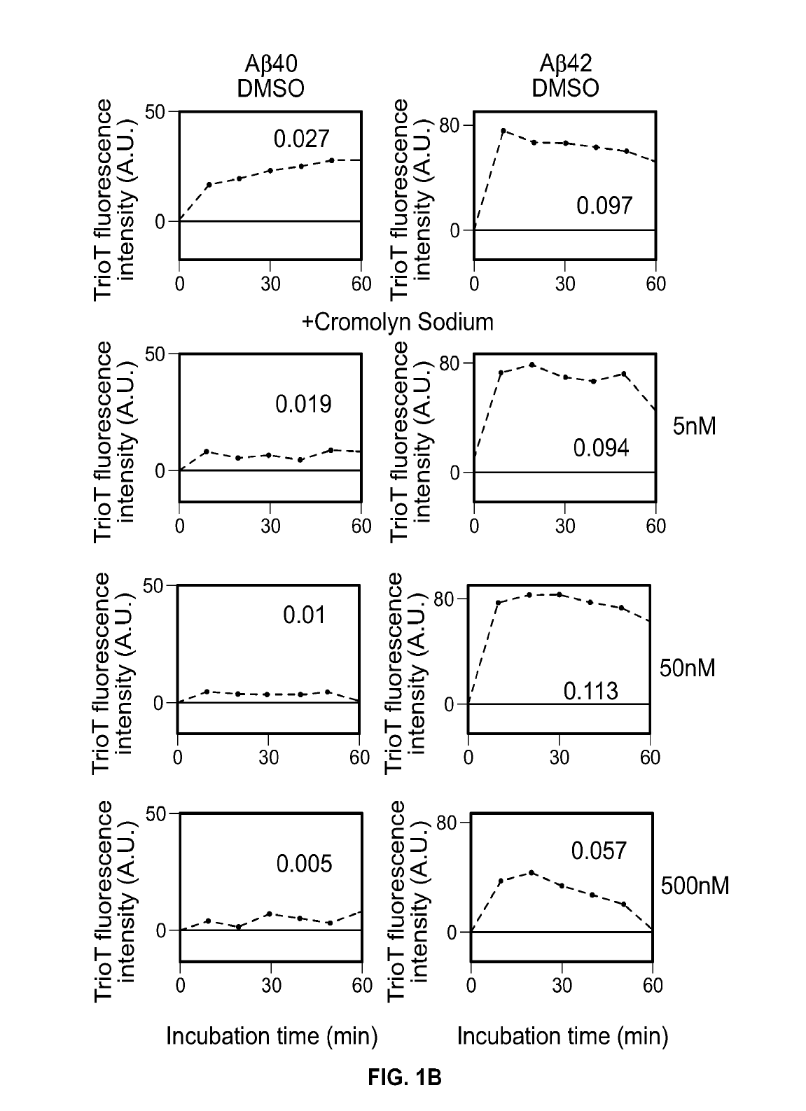

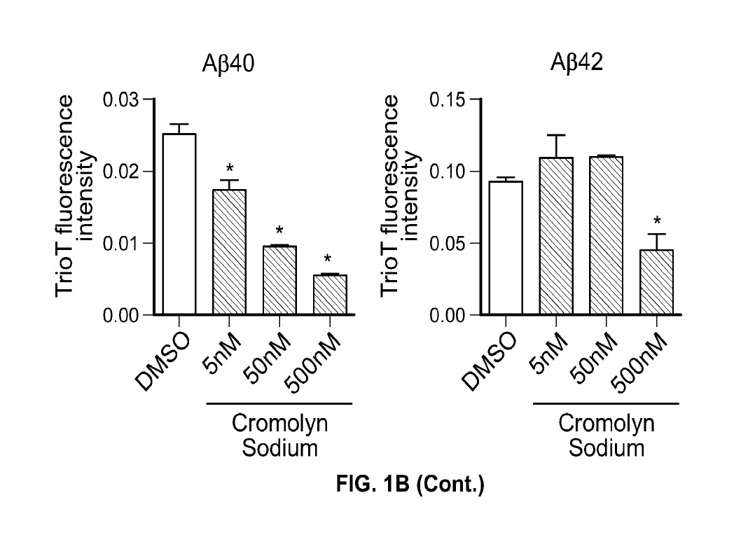

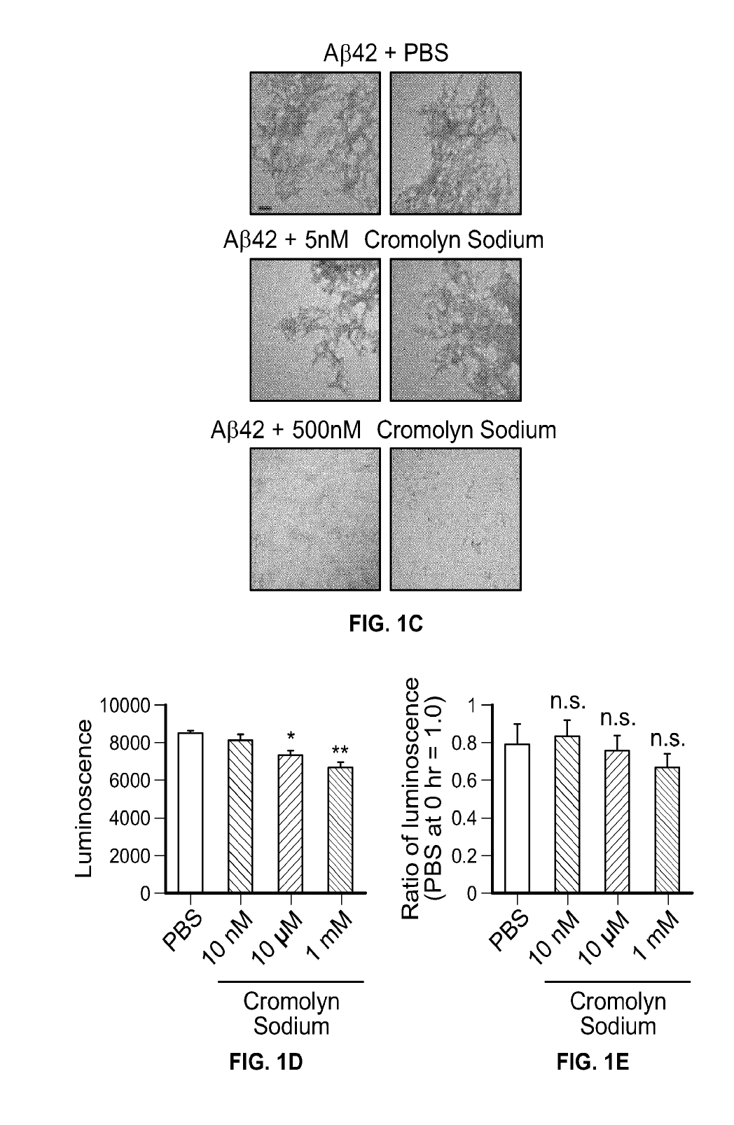

Cromolyn sodium inhibits Aβ polymerization in vitro, but does not impact pre-existing oligomers. The effect of cromolyn sodium on Aβ₄₀ and Aβ₄₂ fibrillization was tested with a thioflavin T assay. Over one hour of incubation at 37° C. with increasing concentrations of cromolyn sodium (5, 50, 5000 nM) inhibited Aβ fibril formation in vitro at a nanomolar concentration (FIG. 1B). Using TEM, the formation of Aβ₄₂ fibrils was inhibited after incubation with 500 nM of cromolyn sodium (FIG. 1C), whereas no effect was detected at a lower concentration (50 nM). Using a split-luciferase complementation method to specifically monitor oligomer formation, treatment of HEK293 cells overexpressing both N- or C-terminal of luciferase conjugated Aβ₄₂ with cromolyn sodium significantly decreased the luminescence signal in a dose-dependent manner. (FIG. 1D). However, this effect could only be detected with concentrations of cromolyn sodium above 10 μM. This discrepancy with the thioflavin-T assay may be due to the fact that our split-luciferase complementation method was performed in a cellular environment. In addition, this oligomerization assay was based on the presence of Aβ₄₂ peptides that are more amyloidogenic and aggregate faster than Aβ₄₀ peptides. By contrast, addition of cromolyn sodium to conditioned media that already contained pre-existing oligomers failed to impact the luminescence signal (FIG. 1E). These data indicate that cromolyn sodium efficiently prevented Aβ polymerization into higher ordered oligomers or fibrils, but cannot dissociate pre-existing aggregates.

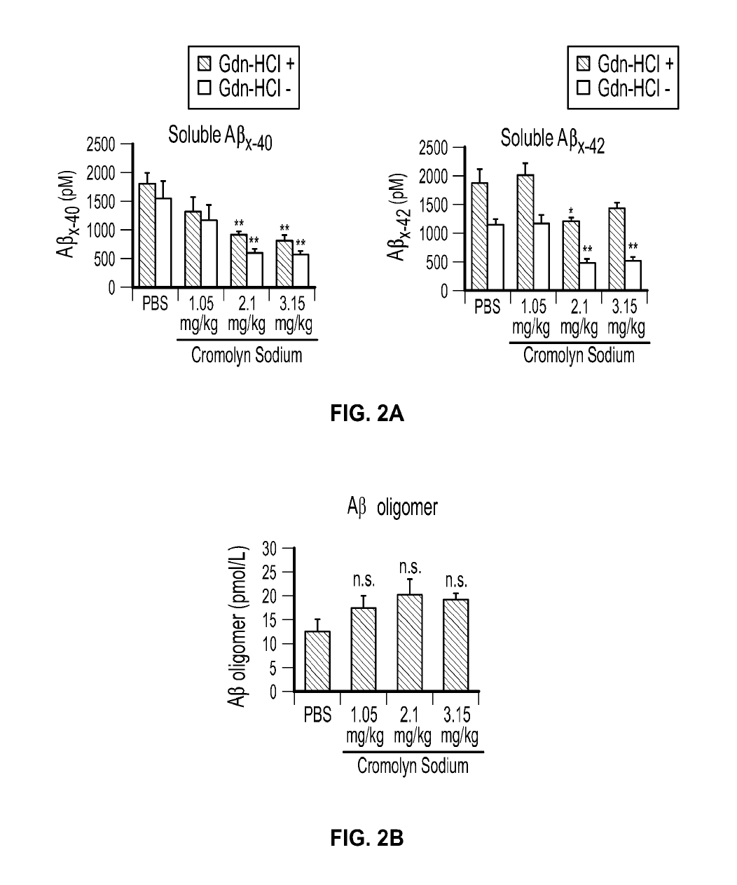

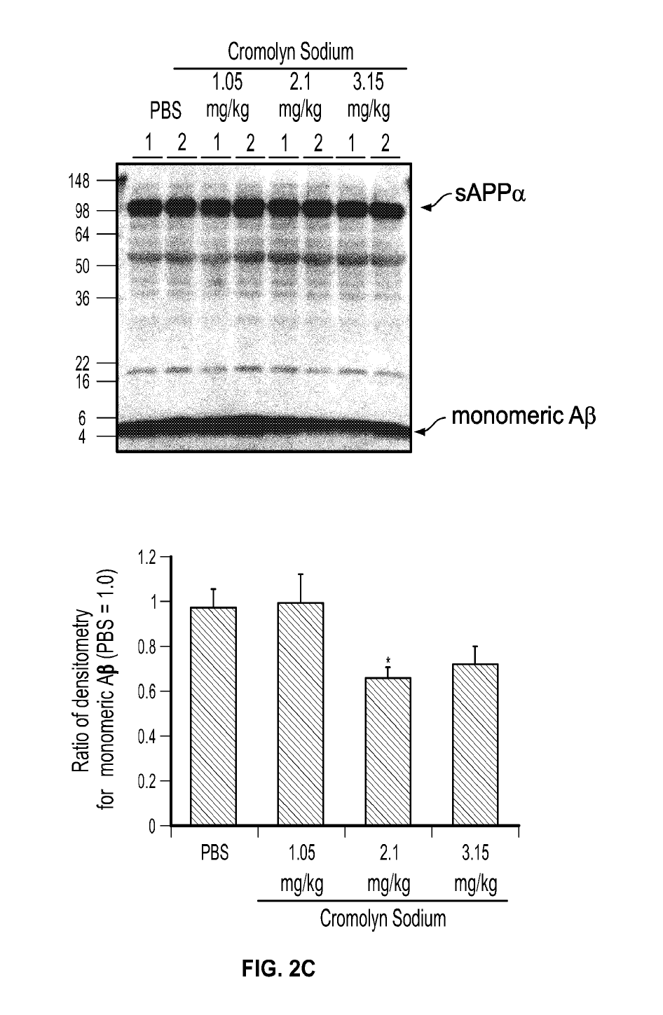

One week exposure with cromolyn sodium in APP/PSI mice significantly lowered the content of soluble Aβ in vivo, but does not affect amyloid deposition or highly fibrillar Aβ species. Cromolyn sodium interfered with Aβ aggregation processes in vitro and therefore may be classified as an anti-amyloidogenic compound. Acute exposure of AD transgenic mice with 2.1 mg/kg or 3.15 mg/kg cromolyn sodium for seven days significantly lowered the content of both TBS-soluble Aβₓ₋₄₀ and Aβₓ₋₄₂ by more than 50% (2.1 mg/kg dose: 39.5% for Aβₓ₋₄₀, 40.9% for Aβₓ₋₄₂; 3.15 mg/kg dose: 37.1% for Aβₓ₋₄₀ 46.2% for Aβₓ₋₄₂ respectively) (FIG. 2A).

TBS soluble fractions were incubated with 0.5 M guanidine (Gdn-HCl) at 37° C. for 30 min to dissociate oligomers or other complexes formed between Aβ and other proteins. The levels of Aβ after incubation generally increased compared with native conditions, especially Aβₓ₋₄₂ that is more prone to aggregation. Treatment with cromolyn sodium lowered the total level of TBS soluble Aβ in a dose-dependent manner (2.1 mg/kg dose: 50.7% for Aβₓ₋₄₀, 63.3% for Aβₓ₋₄₂; 3.15 mg/kg dose: 44.6% for Aβₓ₋₄₀ 76.1% for Aβₓ₋₄₂ respectively) (FIG. 2A).

Cromolyn sodium did not significantly alter the content of higher-order amyloid species. In order to further examine this result, the concentrations of Aβ oligomers were also specifically measured using the 82E1/82E1 ELISA assay that uses the same capture and detection antibody. Again, no changes in the levels of oligomeric aggregates could be detected (FIG. 2B). TBS soluble extracts were also subjected to SDS-PAGE. Quantification of the 4 kDa Aβ band using 6E10 and 82E1 detection antibodies showed that cromolyn sodium decreased the amounts of monomeric Aβ (FIG. 2C), confirming the initial ELISA data. Because of the low proportion of soluble Aβ oligomers as compared with the total levels of Aβ, and did not detect those specific aggregates by western blotting.

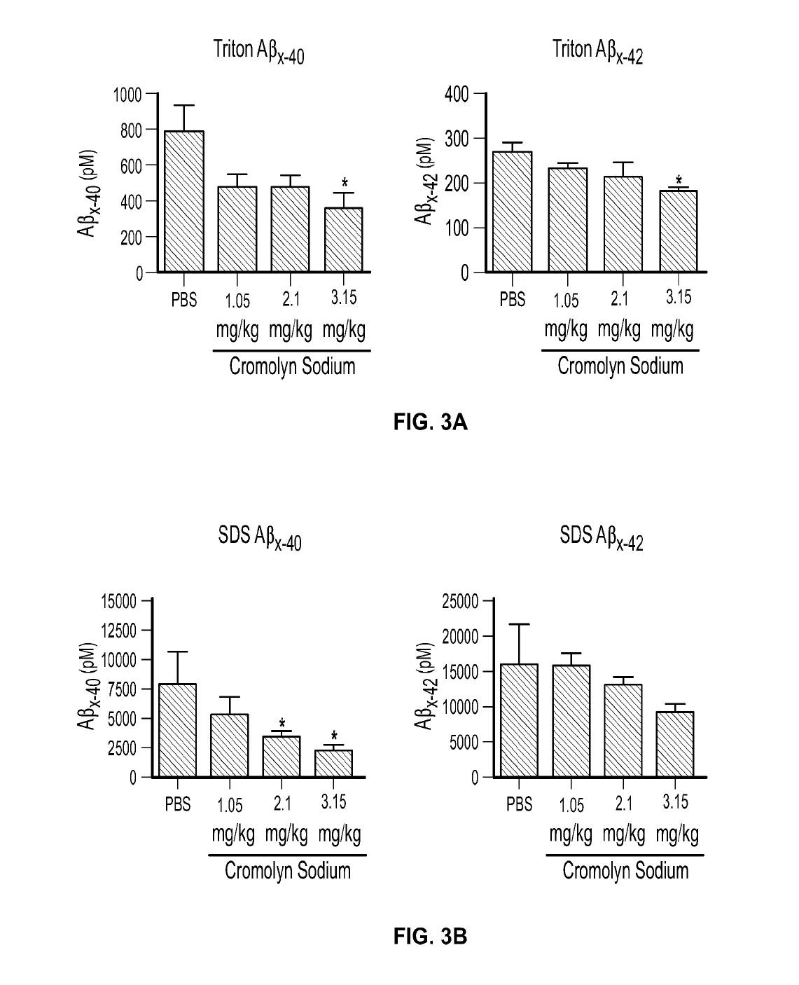

Concentrations of Aβ detergent resistant species sequentially extracted in 2% triton (FIG. 3A) and 2% SDS (FIG. 3B) buffers indicated that treatment with the highest does of cromolyn sodium (3.15 mg/kg) significantly decreased the amounts of Aβₓ₋₄₀ and Aβₓ₋₄₂ as compared to PBS controls. Cromolyn sodium appeared to have a large impact in decreasing Aβₓ₋₄₀ than Aβₓ₋₄₂ for all fractions considered.

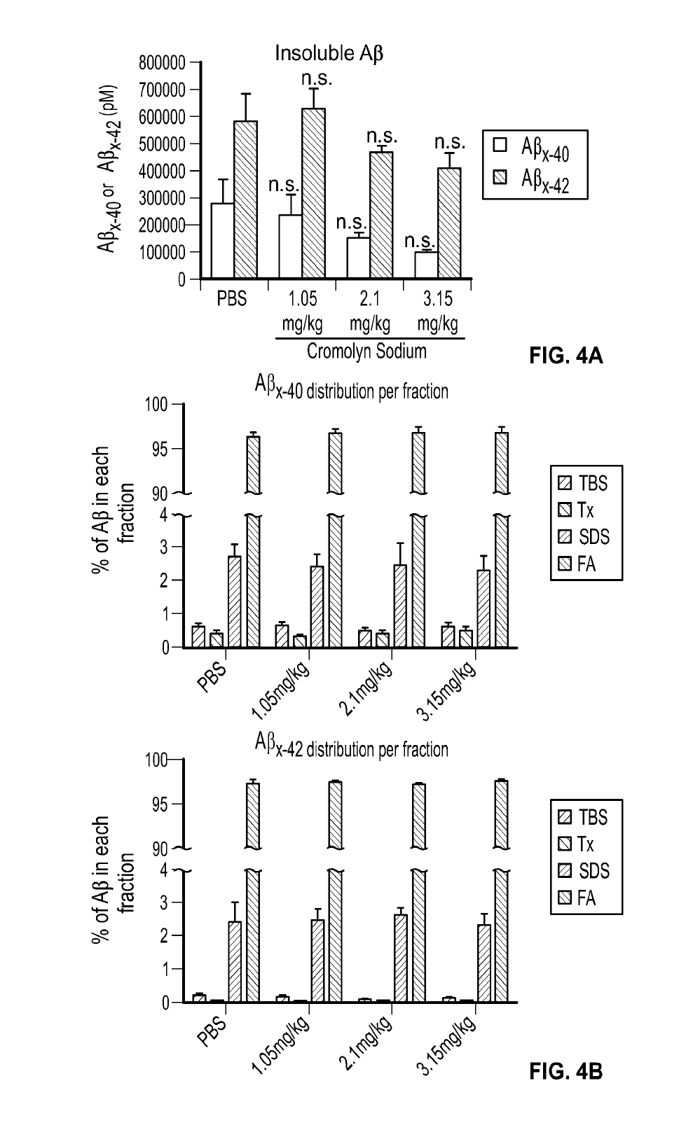

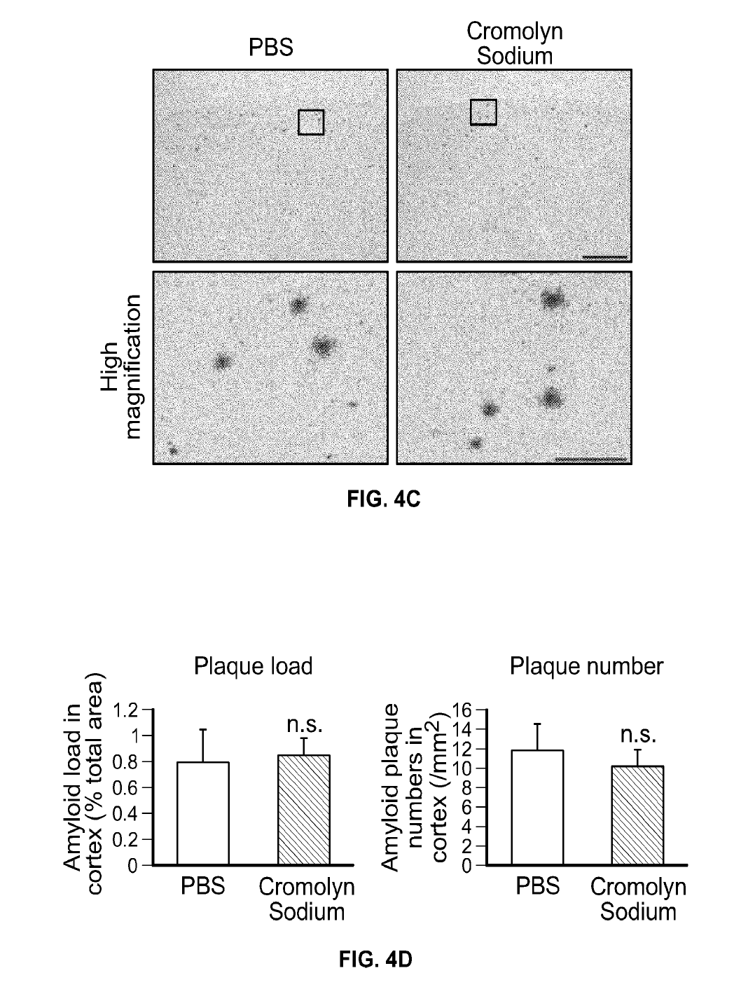

The impact of cromolyn sodium on the most insoluble fraction of Aβ peptides (formic acid extracts) and on the density of amyloid deposits were studied. Insoluble Aβ levels were not affected by acute cromolyn sodium administration (FIG. 4A). Because the levels of insoluble Aβ peptides were much higher as compared with the most soluble fractions and because cromolyn sodium only impacted the soluble pool of Aβₓ₋₄₀ and Aβₓ₋₄₂ in TBS, Triton and SDS extracts, it did not overall alter the distribution of Aβ peptides within each biochemical fraction (TBS, Triton, SDS, and formic acid, FIG. 4B). Additional quantification of the amyloid burden and the density of amyloid deposits, assessed immunohistochemically with an anti-Aβ antibody, confirmed that the amount of extracellular deposited aggregates of amyloid peptides remained unaffected after one week of cromolyn sodium treatment (FIGS. 4C and 4D). The data indicated the cromolyn sodium did not primarily affect the most fibrillar forms of amyloid when administered in AD transgenic mice for a short period of time.

Taken together, the results indicated that acute i.p. administration of cromolyn sodium rapidly decreased the amount of TBS, Triton, and SDS soluble monomeric Aβ in vivo, which constitutes the most exchangeable pool of amyloid within the brain.

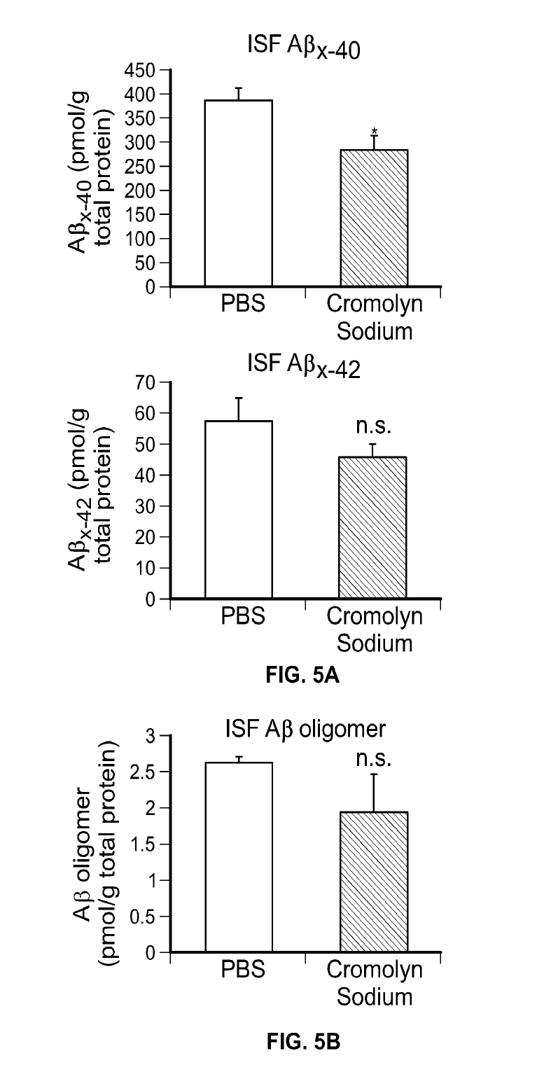

Cromolyn sodium decreased the concentration of Aβ₄₀ in the interstitial fluid of APP/PSI mice. Acute exposure with cromolyn sodium primarily decreased the amount of soluble monomeric amyloid peptides. APP/PSI mice were injected i.p. with PBS or cromolyn sodium at the highest does (3.15 mg/kg body weight) daily for one week. Acute administration of cromolyn sodium dramatically decreased ISF Aβₓ₋₄₀ level by 30% (PBS: 387 pM, cromolyn 283 pM). Both ISF Aβₓ₋₄₂ and Aβ oligomers performed similarly (FIGS. 5A and 5B).

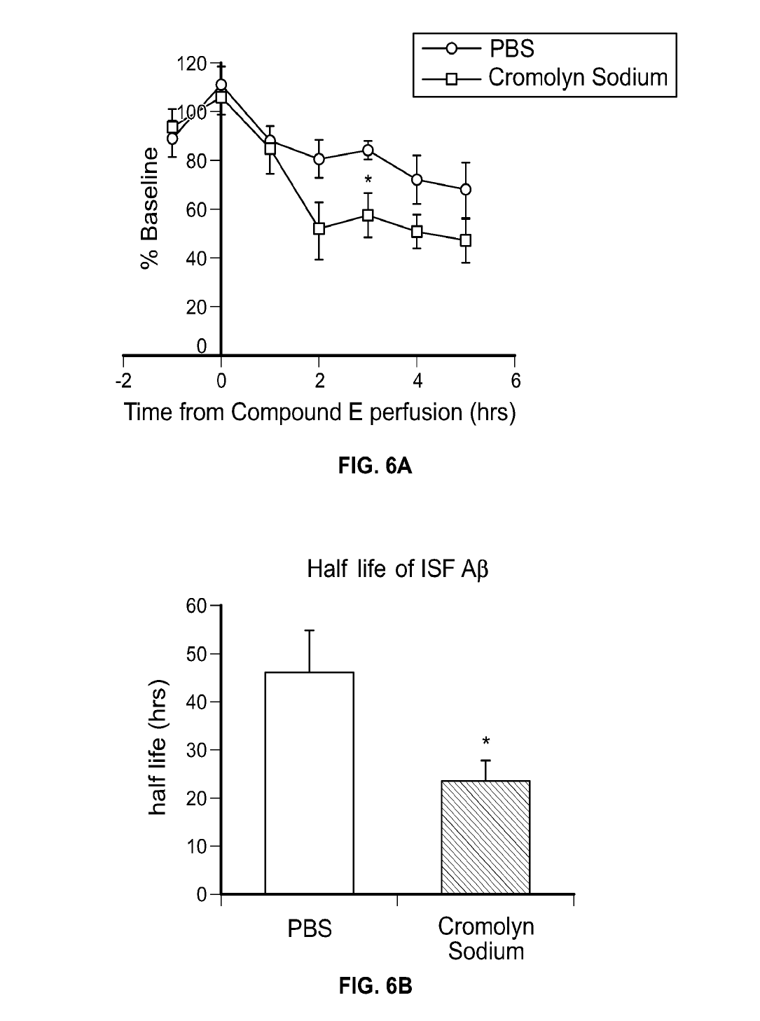

Cromolyn sodium reduced the half-life of Aβ within ISF, a process related to microglial uptake rather than egress of Aβ through the blood brain barrier. The half-life of Aβ in ISF was estimated using reverse microdialysis with the γ-secretase inhibitor Compound E. Mice were treated at the highest dose (3.15 mg/kg body weight). In mice injected with cromolyn sodium ISF Aβ levels started to decrease only 2 hours after administration of Compound E, significantly faster than in PBS treated mice. (FIG. 6A). When calculated, the half-life of ISF Aβ in cromolyn sodium treated mice was shorter than control by about 50% (FIG. 6B), indicating that ISF Aβ was more rapidly cleared after treatment with this compound.

What is claimed is:

1. A method of treating Alzheimer's Disease comprising administering to a subject in need thereof a therapeutically effective amount of cromolyn.

2. The method according to claim 1, wherein the cromolyn is cromolyn sodium.

3. The method according to claim 1 further comprising administering ibuprofen.

4. The method according to claim 1, wherein cromolyn is administered to 17.1 mg.

5. The method according to claim 3, wherein ibuprofen is administered in an amount of 10 mg.

6. The method according to claim 1, wherein the cromolyn is delivered orally, via inhaler, intravenously, intraperitoneally, or transdermally.

7. The method according to claim 1, wherein the effective amount of cromolyn decreased Aβ by about 10% to about 50% after one week of treatment.

8. The method according to claim 1, wherein the cromolyn is administered to achieve a cromolyn concentration in plasma of about 14-133 ng/ml.

9. The method according to claim 1, wherein the cromolyn is administered to achieve a cromolyn concentration in plasma of about 46 ng/ml.

10. The method according to claim 8, wherein the cromolyn concentration in plasma is achieved at about 6-60 minutes.

11. The method according to claim 8, wherein the cromolyn concentration in plasma is achieved in about 22 minutes.

12. The method according to claim 1, wherein the cromolyn achieves an average Cₘₐₓ cromolyn concentration in the CSF of about 0.3 to about −04 ng/ml.

13. The method according to claim 1, wherein the cromolyn achieves an average Cₘₐₓ cromolyn concentration in the CSF of about 0.24 ng/ml.

14. The method according to claim 3, wherein the ibuprofen achieves an average Cₘₐₓ in the CSF of about 2.3 to 5.2 g/nl.

15. The method according to claim 3, wherein the ibuprofen achieves an average Cₘₐₓ in the CSF of about 3.94 g/nl.

16. The method according to claim 14, wherein the ibuprofen Cₘₐₓ is achieved in about 2-4 hours.

17. The method according to claim 14, wherein the ibuprofen Cₘₐₓ is achieved in about 2.55 hours.

18. The method according to claim 3, wherein the ibuprofen achieves an average Cₘₐₓ ibuprofen concentration in plasma of about 25 to about 1970 ng/ml.

19. The method according to claim 3, wherein the ibuprofen achieves an average Cₘₐₓ ibuprofen concentration in plasma of about 1091 ng/ml.
